(19) United States
(12) Patent Application Publication
(10) Pub. No.: US 2002/0173453 A1
(43) Pub. Date: Nov. 21, 2002

(54) Method of treating renal injury

(76) Inventors: Rama Akella, Austin, TX (US); John Paul Ranieri, Austin, TX (US)

Correspondence Address: Timothy L. Scott, Senior Patent Counsel, SULZER MEDICA USA INC., Suite 1600, 3 East Greenway Plaza, Houston, TX, 77046, US

(21) Appl. No.: 09/738,524

(22) Filed: Dec. 15, 2000

(51) Int. Cl. A61K 38/18

(52) U.S. Cl. 514/12

(57) ABSTRACT

Herein is disclosed a method of treating renal injury in a mammal, comprising administering to the mammal a mixture of growth factors comprising at least two selected from bone morphogenic protein-2 (BMP-2), bone morphogenic protein-3 (BMP-3), bone morphogenic protein-4 (BMP-4), bone morphogenic protein-5 (BMP-5), bone morphogenic protein-6 (BMP-6), bone morphogenic protein-7 (BMP-7), transforming growth factor β (TGF-β1, transforming growth factor β (TGF-β2, transforming growth factor β3. (TGF-β3, or fibroblast growth factor 1 (FGF-1).

### BACKGROUND OF THE INVENTION

The present invention relates generally to the field of treating renal injury. More particularly, it concerns the treatment of renal injury by the administration of a mixture of bone-derived growth factors. The mixture of growth factors may comprise BMP-2, BMP-3, BMP-4, BMP-5, BMP-6, BMP-7, TGF-β1, TGF-β2, TGF-β3, and FGF-1 .

“Renal injury,” as the term is used herein, refers to a state of impaired kidney function. Impaired kidney function can be identified from a reduced glomerular filtration rate, an increased serum creatinine concentration, an increased blood urea nitrogen (BUN) concentration, or other symptoms recognizable by persons of skill in the art. “Renal injury” is not limited to impaired kidney function caused by physical trauma to the kidney, and can include, for example, physical trauma, sepsis, exposure to toxic compounds, exposure to medicinal drugs, or tumor growth in or metastasis to the kidney, among others.

“Treating” renal injury, therefor refers to a reduction in the impairment of kidney function, or minimizing a future impairment of kidney function if administered prophylactically. Reduced impairment of kidney function, or minimization of impairment, can be identified by the criteria set forth above, e.g., glomerular filtration rate, the serum creatinine concentration, blood urea nitrogen concentration, or alleviation of other symptoms recognizable by persons of skill in the art. Acute renal failure is a life threatening type of renal injury and, in terms of treatment costs, is the most costly kidney disease. The mortality rate associated with acute renal failure is extremely high and is commonly a result of progression of the disorder to end stage renal disease. This high mortality rate persists despite recent advances in supportive care. End stage renal disease currently afflicts roughly 280,000 people in the U.S., and leads to approximately 50,000 deaths each year.

Currently, two of the leading treatments for acute renal failure are dialysis or kidney transplantation, neither of which is an acceptable long-term solution for the patient group. Dialysis, with an annual mortality rate of about 25%, is clearly an undesirable treatment method. In addition to its high mortality rate it is inconvenient and uncomfortable to the patient. However, it is for many patients the only available treatment option. The survival rate for kidney transplant patients at 5 years is in the range of 90-95%. However, transplants are limited by the availability of donor organs, the operative risks associated with major surgery, and the post-operative requirement of taking immunosuppressant drugs to prevent rejection of the transplanted kidney, thereby increasing the patient's risk of secondary and/or opportunistic infection or disease.

In some instances, however, near-total recovery after acute renal failure does occur, indicating that regeneration of damaged renal tissue is possible. Regeneration is characterized by rapid proliferation of damaged epithelial cells that line the tubules of the kidney. As a result, methodologies to assist regeneration of damaged epithelium are being pursued. These methodologies, however, are primarily indirect treatments, e.g. fluid and electrolyte therapy, or temporary dialysis and withdrawal of the agent that inflicted the renal injury.

The growth factors BMP-7 and IGF-1 have been examined in terms of their role in the renal tissue regenerative process. BMP-7 (bone morphogenic protein 7, also known as OP-1) is known to play a role in embryonic renal morphogenesis, by inducing metanephric mesenchyme differentiation. Preclinical trials undertaken by Hruska's group at the Washington University School of Medicine have shown that administration of BMP-7 preserves kidney function in models of acute renal failure, and also enhances filtration and blood flow (BW Healthwire, Nov. 8, 1999; presented at the 1999 Annual Meeting of the American Society of Nephrology).

IGF-1 (insulin-like growth factor 1) is expressed in healthy kidneys. Shortly after induction of ischemic acute renal injury, expression of IGF-1 increased in proximal tubules and remained elevated for at least 7 days. However, two clinical studies involving recombinant human IGF-1 (rhIGF-1) proved inconclusive (Bohe et al., Nephrologie 19:1, 11-13 (1998); Hirschberg et al., Kidney int. 55:6, 2423-2432 (1999).

Other growth factors which have been shown to have receptors expressed by proximal tubular renal cells, to induce proliferation of proximal tubular cells in vitro, or are otherwise believed to play a role in kidney regeneration, include EGF (epidermal growth factor), HGF (hepatocyte growth factor), TGF-α, TGF-β(transforming growth factor α, β), PDGF (platelet-derived growth factor), and FGF (fibroblast growth factor).

It is desirable to treat renal injury by the administration of a growth factor or factors. Preferably, improvement in kidney function brought about by the treatment will be superior to that brought about by techniques known in the art. It is desirable for the growth factor or factors to be readily purified from convenient starting materials.

### SUMMARY OF THE INVENTION

In one embodiment, the present invention relates to compositions useful for treating renal injury in a mammal, comprising a mixture of growth factors comprising at least two growth factors selected from BMP-2, BMP-3, BMP-4, BMP-5, BMP-6, BMP-7, TGF-β1, TGF-β2, TGF-β3, or FGF-1. In a preferred embodiment, the mixture comprises BMP-2, BMP-3, BMP-4, BMP-5, BMP-6, BMP-7, TGF-β1, TGF-β2, TGF-β3, and FGF-1.

In another embodiment, the present invention provides methods for treatment of renal injury, comprising administering to a mammal a mixture of growth factors comprising at least two growth factors selected from BMP-2, BMP-3, BMP-4, BMP-5, BMP-6, BMP-7, TGF-β1, TGF-β2, TGF-β3, or FGF-1. Preferably, the mixture can be administered subcutaneously, intramuscularly, or intravascularly. Preferably, the mammal is a human. The method is at least about as effective as methods previously known in the art, with the potential to be more effective than prior art approaches as a result of synergism between various growth factors in the mixture. The mixture can be prepared using recombinant techniques, or can be purified from convenient, available starting materials such as bovine bone.

### BRIEF DESCRIPTION OF THE DRAWINGS

FIG. 1 illustrates an SDS-PAGE of a protein mixture useful in the present invention, both in reduced and nonreduced forms.

FIG. 2 is an SDS-PAGE gel of HPLC fractions 27-36 of a protein mixture according to an embodiment of the present invention.

FIG. 3 is an SDS-PAGE gel with identified bands indicated according to the legend of FIG. 4.

FIG. 4 is an SDS-PAGE gel of a protein mixture according to an embodiment of the present invention with identified bands indicated, as provided in the legend.

FIG. 5 is two dimensional (2-D) SDS-PAGE gel of a protein mixture according to an embodiment of the present invention with internal standards indicated by arrows.

FIG. 6 is a 2-D SDS-PAGE gel of a protein mixture according to an embodiment of the present invention with circled proteins identified as in the legend.

FIGS. 7A-O are mass spectrometer results for tryptic fragments from one dimensional (1-D) gels of a protein mixture according to an embodiment of the present invention.

FIG. 8 is a 2-D gel Western blot of a protein mixture according to an embodiment of the present invention labeled with anti-phosphotyrosine antibody.

FIGS. 9A-D are 2-D gel Western blots of a protein mixture according to an embodiment of the present invention, labeled with indicated antibodies. FIG. 9A indicates the presence of BMP-3 and BMP-2. FIG. 9B indicates the presence of BMP-3 and BMP-7. FIG. 9C indicates the presence of BMP-7 and BMP-2, and FIG. 9D indicates the presence of BMP-3 and TGF-β1.

FIG. 10 is a PAS (periodic acid schiff) stained SDS-PAGE gel of HPLC fractions of a protein mixture according to an embodiment of the present invention.

FIG. 11 is an anti-BMP-7 stained SDS-PAGE gel of a PNGase F treated protein mixture according to an embodiment of the present invention.

FIG. 12 is an anti-BMP-2 stained SDS-PAGE gel of a PNGase F treated protein mixture according to an embodiment of the present invention.

FIGS. 13A-B are bar charts showing explant mass of glycosylated components in a protein mixture according to an embodiment of the present invention (FIG. 13A) and ALP score (FIG. 13B) of the same components.

FIG. 14 is a chart showing antibody listing and reactivity.

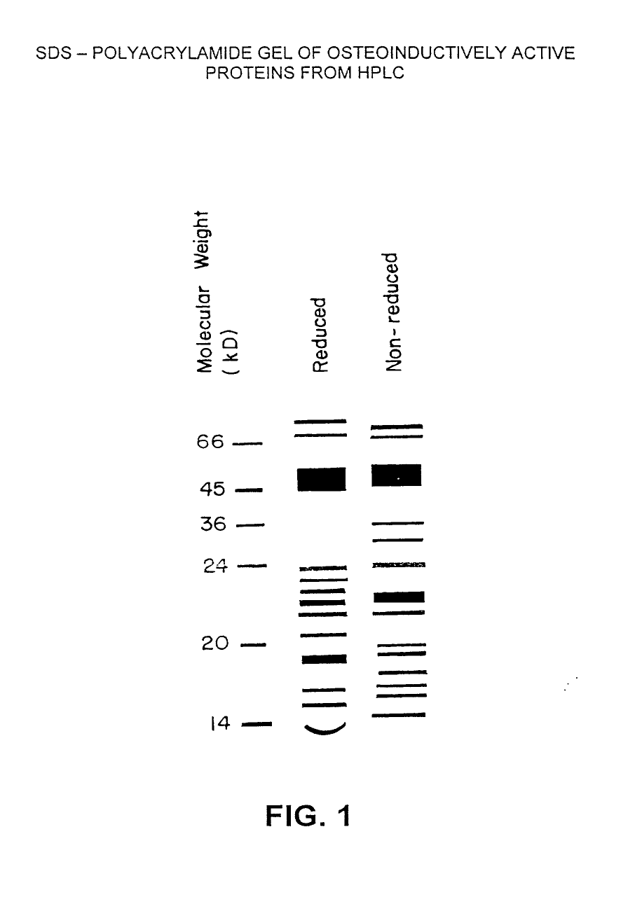

FIGS. 1 5A-B together comprise a chart showing tryptic fragment sequencing data for components of a protein mixture according to an embodiment of the present invention.

FIGS. 16A-F together comprise a chart showing tryptic fragment mass spectrometry data for components of a protein mixture according to an embodiment of the present invention.

FIGS. 17A-B are an SDS-gel (FIG. 17B) and a scanning densitometer scan (FIG. 17A) of the same gel for a protein mixture according to an embodiment of the present invention.

FIG. 18 is a chart illustrating the relative mass, from scanning densitometer quantification, of protein components in a protein mixture according to an embodiment of the present invention.

FIGS. 19A-D together comprise a chart showing mass spectrometry data of various protein fragments from 2D gels of a protein mixture according to an embodiment of the present invention.

### DESCRIPTION OF ILLUSTRATIVE EMBODIMENTS

In one embodiment, the present invention relates to a method of treating renal injury in a mammal, comprising administering to the mammal a mixture of growth factors comprising at least two selected from bone morphogenic protein-2 (BMP-2), bone morphogenic protein-3 (BMP-3), bone morphogenic protein-4 (BMP-4), bone morphogenic protein-5 (BMP-5), bone morphogenic protein-6 (BMP-6), bone morphogenic protein-7 (BMP-7), transforming growth factor β1 (TGF-β1, transforming growth factor β2 (TGF-β2, transforming growth factor β3 (TGF-β3, or fibroblast growth factor 1 (FGF-1).

Without being bound by any particular theory, it is believed that “treating” renal injury according to the present method involves the promotion of proliferation, differentiation, or both in renal proximal tubular epithelial cells; the inhibition of a fibrotic response; the regulation of the cell cycle; the inhibition of apoptosis; the assistance of production of extracellular matrix; or some or all of the foregoing.

The method involves the administration of a mixture of growth factor s to the mammal. The mixture of growth factors comprises at least two selected from BMP-2, BMP-3, BMP-4, BMP-5, BMP-6, BMP-7, TGF-β1, TGF-β2, TGF-β3, or FGF-1. “Growth factor” herein refers to a peptide or polypeptide which is capable of inducing cellular proliferation or cellular differentiation of a mammalian cell type either in vitro or in vivo.

The growth factors suitable for use in embodiments of the present invention can be produced by recombinant techniques, or they can be isolated from mammalian tissues. Preferably, the growth factors are isolated from bovine bone, as will be described in more detail below. The proportions of the various growth factors in the mixture can vary.

In addition to the growth factors named and described above, the mixture can comprise additional growth factors. Such additional growth factors can include insulin-like growth factor-1 (IGF-1), epidermal growth factor (EGF), hepatocyte growth factor (HGF), transforming growth factor α (TGF-α, or platelet-derived growth factor (PDGF), among others. However, the presence of additional growth factors is not required.

The mixture may also comprise proteins that are not growth factors. These non-growth factor proteins may be chosen for inclusion in the mixture, or may be present as a side-effect of the purification process. Provided the non-growth factor proteins do not pose harm to the subject mammal, there is no limitation on their inclusion. Typical non-growth factor proteins that may be present in the mixture include lysyl oxidase related proteins (LORP), factor XIII, SPP24, histones (including H1.c and H1.x), and ribosomal proteins (including RS3a, RS20, RL6, and RL32).

The protein mixture may be provided in a buffered aqueous solution suitable for the storage and administration of proteins, although other formulations can be used. The mixture can also comprise preservatives, adjuvants, pharmaceutically-acceptable carriers, or other compounds suitable for storing the growth factors or for administering the growth factors to the mammal. Preferably, any additional growth factors, non-growth factor proteins, buffering agent, preservatives, adjuvants, or other compounds will not impair the stability or interfere with the activity of the recited growth factors, and preferably also will not engender any side effects upon administration to the mammal.

In a preferred embodiment, the mixture comprises BMP-2, BMP-3, BMP-7, a TGF-?, and an FGF. In a particularly preferred embodiment the mixture comprises BMP-2, BMP-3, BMP-4, BMP-5, BMP-6, BMP-7, TGF-β1, TGF-β2, TGF-β3, and FGF-1. Preparation of a particularly preferred embodiment, hereinafter referred to herein as “BP,” is described in U.S. Pat. Nos. 5,290,763, 5,371,191, and 5,563,124 (each of which is hereby incorporated by reference herein in its entirety).

In brief, the BP cocktail is prepared by guanidine hydrochloride protein extraction of demineralized bone particles. The extract solution is filtered, and subjected to a two step ultrafiltration process. In the first ultrafiltration step an ultrafiltration membrane having a nominal molecular weight cut off (MWCO) of 100 kD is employed. The retentate is discarded and the filtrate is subjected to a second ultrafiltration step using an ultrafiltration membrane having a nominal MWCO of about 10 kD. The retentate is then subjected to diafiltration to substitute urea for guanidine. The protein-containing urea solution is then subjected to sequential ion exchange chromatography, first anion exchange chromatography followed by cation exchange chromatography. The osteoinductive proteins produced by the above process are then subjected to HPLC with a preparative VYDAC(tm) column at and eluted with shallow increasing gradient of acetonitrile. One minute fractions of the HPLC column eluate are pooled to make the BP cocktail (fraction number can vary slightly with solvent composition, resin size, volume of production lot, etc.).

One embodiment of the BP cocktail is characterized as shown in FIGS. 1-6. Absolute and relative amounts of the growth factors present in the BP cocktail can be varied by collecting different fractions of the HPLC eluate. In a particularly preferred embodiment, fractions 29-34 are pooled. It is also contemplated that certain proteins may be excluded from the BP mixture without affecting renal injury treatment activity.

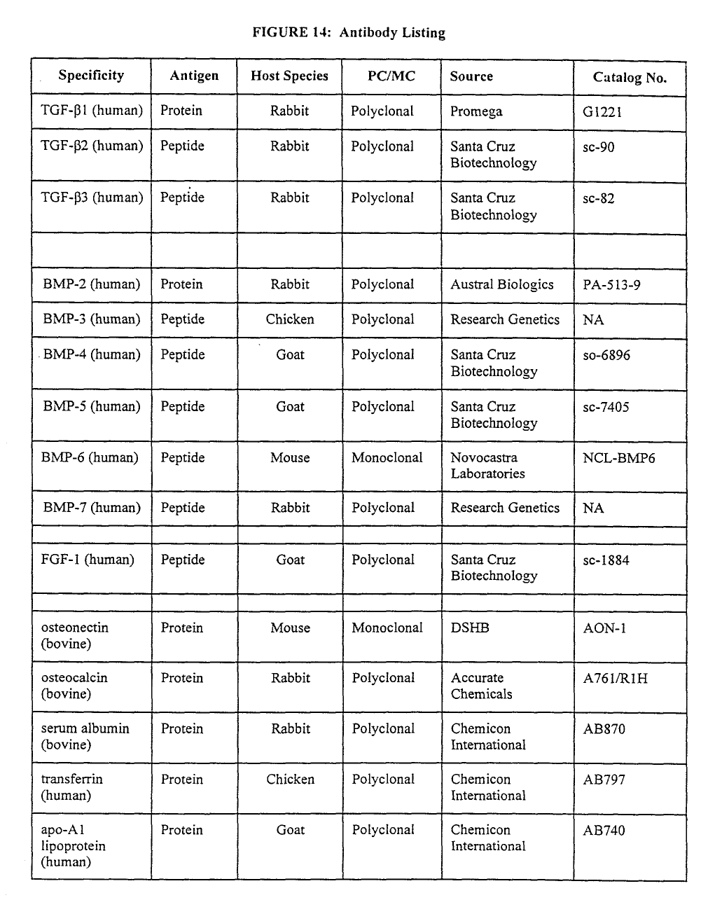

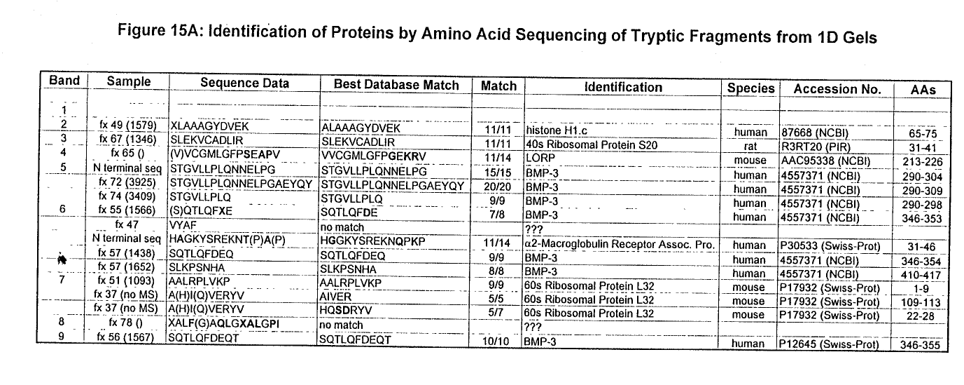

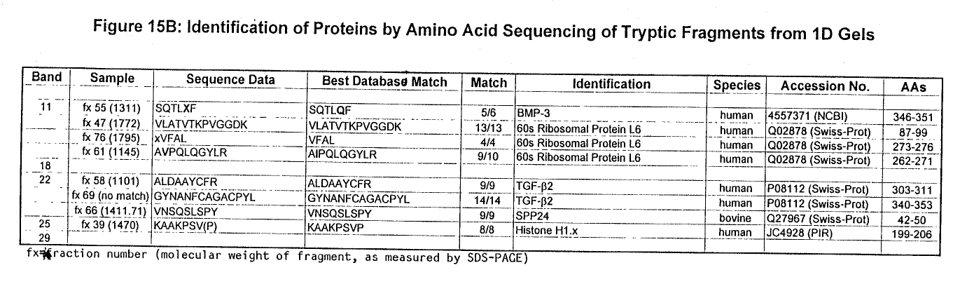

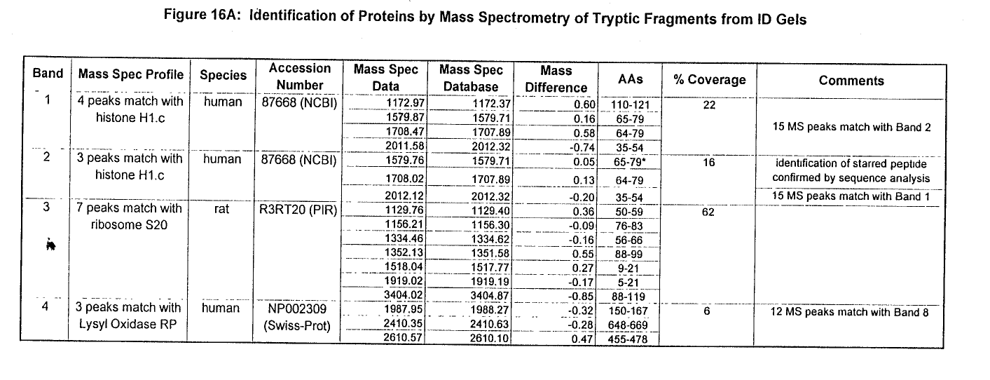

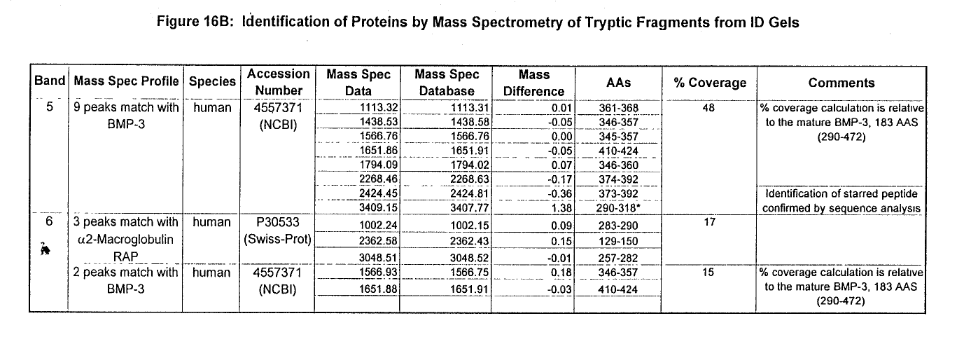

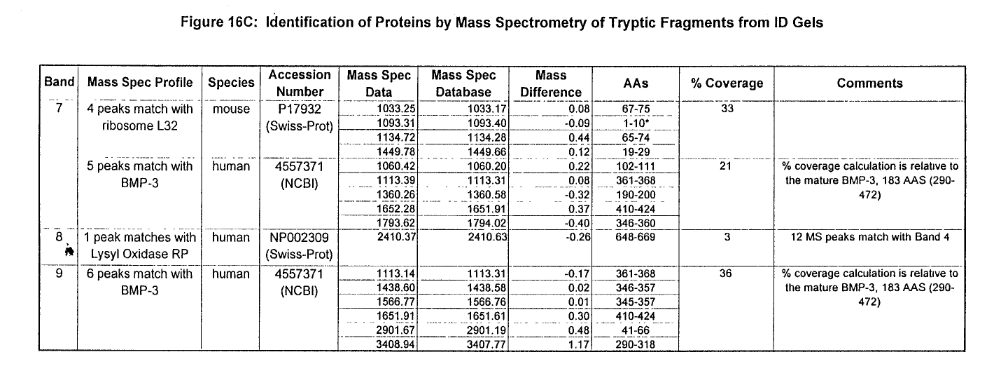

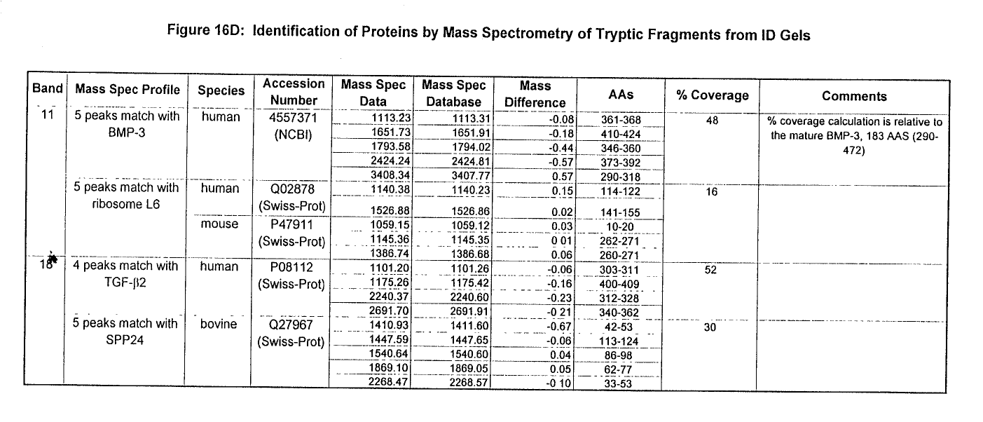

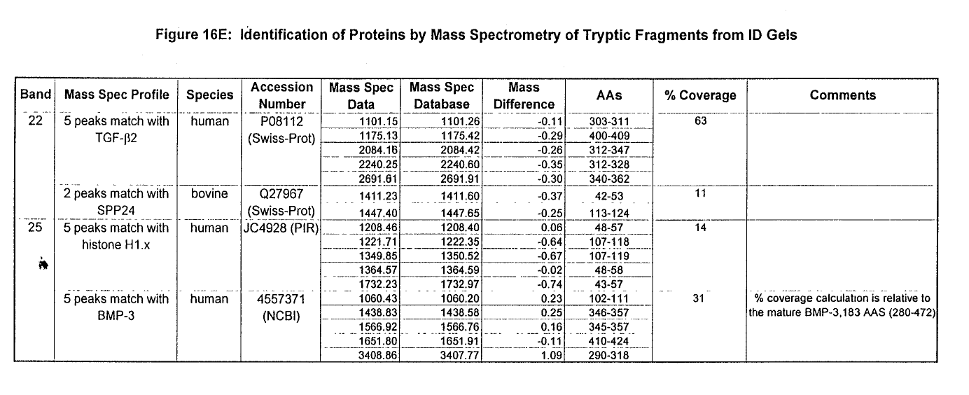

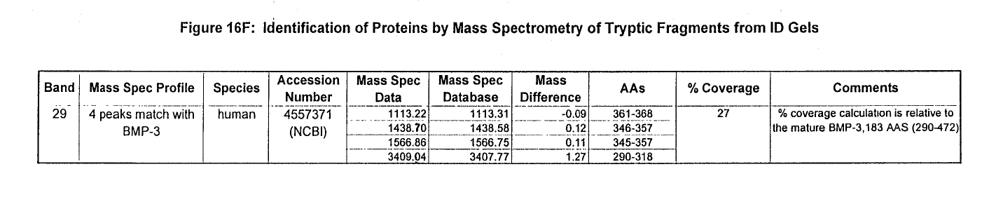

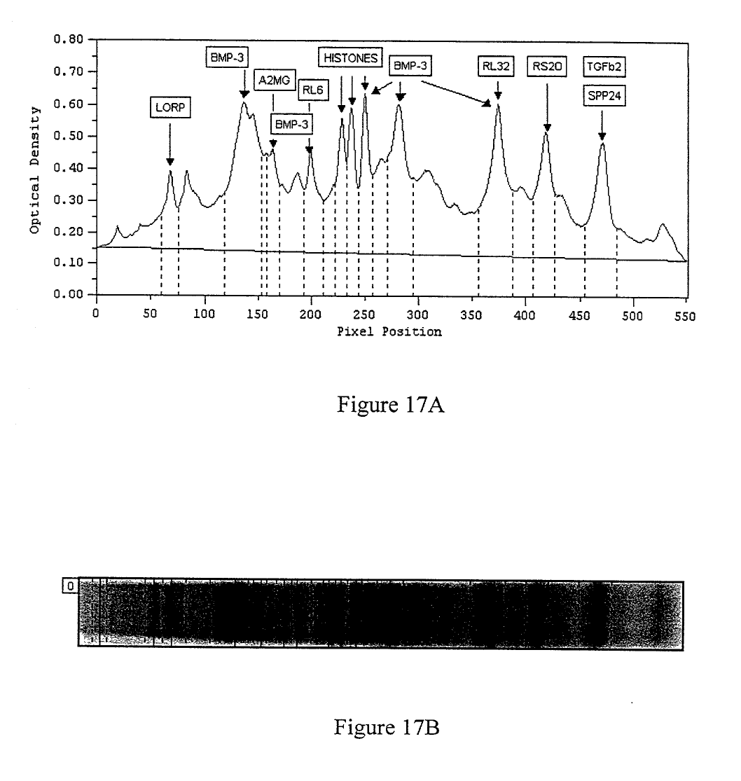

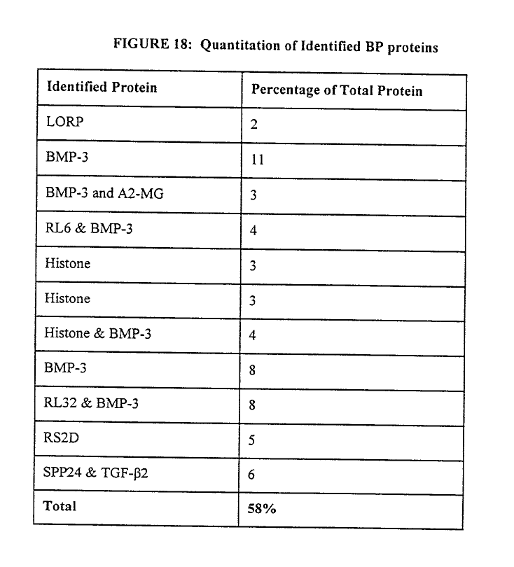

BP was originally discovered as a mixture of proteins having osteogenic activity. However, it contains a plurality of growth factors and subsequent work has revealed it to be strongly angiogenic. In particular, BP contains a number of bone morphogenetic proteins (BMPs), including BMP-2, BMP-3, BMP-4, BMP-5, BMP-6, and BMP-7, as well as TGF-β1, TGF-β2, and TGF-β3. FGF-1 is also present in the mixture. The presence of each of the foregoing proteins was detected using immunoblot techniques, as depicted FIG. 14.

U.S. Pat. Nos. 5,290,763 and 5,371,191 (Poser et al.), and 5,563,124 (Damien et al.) disclose BP derived from bovine bone, although other mammalian bone could be used as a source material. First, the bone is demineralized by grinding bone segments into particles typically less than 4 mm in size, cleaning the bone particles in a detergent solution, and then demineralizing the particles with acid, such as dilute HCl . Other cleaning and demineralizing techniques may also be used. After demineralization, proteins are extracted using a protein denaturant, e.g. guanidinium ion, urea, or both. Extraction temperature is typically less than about 20° C., and extraction duration is typically about 48 hr.

As disclosed for the preparations of Poser et al. and Damien et al., the extracted proteins may be purified by (i) ultrafiltration to separate out high molecular weight proteins, typically with molecular weight cutoff (MWCO) membrane of about 100 kD, (ii) ultrafiltration to separate out low molecular weight proteins, typically with a MWCO membrane of about 10 kD, (iii) transfer, such as by diafiltration or dialysis, to a non-ionic denaturant, e.g. 2M-6M urea buffered with tri[hydroxymethyl]aminomethane (“tris”) and adjusted to about pH 8.5, (iv) an anion exchange process, such as using a quaternary amine resin (e.g. “Q-Sepharose,” Pharmacia) and an eluant comprising 6M urea buffered with tris and 0.10M-0.16M NaCl, (v) a cation exchange process, such as using a sulfonic acid resin (e.g. “S-Sepharose,” Pharmacia) and an eluant comprising urea and 0.6M-1.5M NaCl, and (vi) a reverse phase HPLC process. Although the mixture will typically be purified by a process comprising an ion exchange step, other purification techniques may be employed to obtain purified mixtures of proteins consistent with the present inventions.

Purified BP prepared according to the process disclosed by Poser et al. and Damien et al. has been demonstrated to exhibit osteoinductive activity at about 3 μg when deposited on a suitable carrier and implanted subcutaneously. Upon hydrolysis, the amino acid composition of BP has been shown to be about 23.4 mole % ASP(+ASN) and GLU(+GLN); about 13.5 mole % SER and THR; about 40.0 mole % ALA, GLY, PRO, MET, VAL, ILE, and LEU; about 6.8 mole % TYR and PHE; and about 16.6 mole % HIS, ARG, and LYS.

Specific growth factors present in BP have been identified by partial characterization of BP. For this work, HPLC fractions (one minute intervals) were denatured, reduced with DTT (dithiothreitol), and separated by sodium dodecyl sulfate polyacrylamide gel electrophoresis (SDS-PAGE). Size standards (ST) of 14, 21, 31, 45, 68 and 97 kDa were obtained as Low Range size standards from BIORADTM. In the usual protocol, HPLC fractions 29 through 34 were pooled to produce BP.

An SDS-PAGE gel of BP was also analyzed by Western immunoblot with a series of antibodies: polyclonal rabbit anti-TGF-β1 (human) (Promega, catalog no. G1221); polyclonal rabbit anti-TGF-β2 (human) (Santa Cruz Biotechnology, catalog no. sc-90); polyclonal rabbit anti-TGF-β3 (human) (Santa Cruz Biotechnology, catalog no. sc-82); polyclonal rabbit anti-BMP-2 (human) (Austral Biologics, catalog no. PA-513-9); polyclonal chicken anti-BMP-3 (human) (Research Genetics, catalog no. not available); polyclonal goat anti-BMP-4 (human) (Santa Cruz Biotechnology, catalog no. sc-6896); polyclonal goat anti-BMP-5 (human) (Santa Cruz Biotechnology, catalog no. sc-7405); monoclonal mouse anti-BMP-6 (human) (Novocastra Laboratories, catalog no. NCL-BMP6); polyclonal rabbit anti-BMP-7 (human) (Research Genetics, catalog no. not available); polyclonal goat anti-FGF-1 (human) (Santa Cruz Biotechnology, catalog no. sc-1884); monoclonal mouse anti-osteonectin (bovine) (DSHB, catalog no. AON-1); polyclonal rabbit anti-osteocalcin (bovine) (Accurate Chemicals, catalog no. A761/R1H); polyclonal rabbit anti-serum albumin (bovine) (Chemicon International, catalog no. AB870); polyclonal chicken anti-transferrin (human) (Chemicon International, catalog no. AB797); and polyclonal goat anti-apo-A1 lipoprotein (human) (Chemicon International, catalog no. AB740). Visualization of antibody reactivity was by horseradish peroxidase conjugated to a second antibody and using a chemiluminescent substrate.

BP was further characterized by 2-D (two dimensional) gel electrophoresis. The proteins were separated in the horizontal direction according to charge (pI) and in the vertical direction by size according to the method of O'Farrell et al. (Cell, 12:1133-1142, 1977). Internal standards, specifically tropomyosin (33 kDa, pI 5.2) and lysozyme (14.4 kDa, pI 10.5-11.0), were included and the 2-D gel was visualized by Coomassie blue staining. The proteins were identified by mass spectrometry and amino acid sequencing of tryptic peptides, as described below. Proteins identified included factor XIII, RL3, TGF-β2, SPP24, lysyl oxidase related proteins (LORP), BMP-3, cathepsin L, and RS3a.

The various components of BP were characterized by mass spectrometry and amino acid sequencing of tryptic fragments where there were sufficient levels of protein for analysis. The major bands in the 1 -D (one dimensional) gels were excised, eluted, subjected to tryptic digestion, purified by HPLC and sequenced by methods known in the art. The major bands identified were histone Hi.c, RS20, LORP, BMP-3, α2 macroglobulin receptor associated protein, RL6, TGF-β2, SPP 24, factor H, TGF-β2, histone H1.x, and RL32. The sequence data was compared against known sequences, and the fragments were identified. In some cases, the identification was tentative due to possible variation between known human sequences and the bovine sequences present in BP, or possible posttranslational modifications, as discussed below.

The same tryptic protein fragments were analyzed by mass spectrometry. With the exception of factor H, the major bands identified by sequencing were confirmed, with the caveat that assignment of band identity may be tentative based on species differences and posttranslational modifications.

The identified components of BP were quantified by a scanning densitometer scan of a stained SDS-PAGE gel of BP. The identified proteins were labeled and quantified by measuring the area under the curve. The following identifications, and percentages of total protein, were made: LORP, 2%; BMP-3, 19%; BMP-3 and/or α2 macroglobulin receptor associated protein, 3%; BMP-3 and/or RL6, 4%; histones, 6%; histone and/or BMP-3, 4%; RL32 and/or BMP-3, 8%; RS20, 5%; SPP24 and/or TGF-β2, 6%. Identified proteins comprised 58% of the total. In addition, TGF-≯1 was quantified using commercially pure TGF-≯1 as a standard, and was determined to represent less than 1% of BP.

The identified proteins fell roughly into three categories: ribosomal proteins, histones, and growth factors, including active growth factors comprising members of the TGF-≯ superfamily of growth factors, which includes the bone morphogenic proteins (BMPs). It is believed that the ribosomal proteins and histone proteins may be removed from the BP without loss of activity, and the specific activity is expected to increase correspondingly.

Because several of the proteins migrated at more than one size (e.g., BMP-3 migrated as 5 bands), investigations were undertaken to investigate the extent of posttranslational modification of BP components. Phosphorylation was measured by anti-phosphotyrosine immunoblot (such as by 2-D electroblot using, e.g., phosphotyrosine mouse monoclonal antibody (Sigma, catalog no. A-5964)) and by phosphatase studies. Several proteins were thus shown to be phosphorylated at one or more tyrosine residues.

Similar 2-D electroblots were probed with BP component specific antibodies. The filters were probed with antibodies against, and indicated the presence of, BMP-2, BMP-3, BMP-7, and TGF-≯1. Each showed the characteristic, single-size band migrating at varying pI, as is typical of a protein existing in various phosphorylation states.

Native and phosphatase treated BP samples were also assayed for morphogenic activity by explant mass and ALP (alkaline phosphatase) score. The results showed that BP treatment reduces the explant mass and ALP score from 100% to about 60%.

BP was also analyzed for glycosylation, such as by staining with periodic acid schiff (PAS)—a non-specific carbohydrate stain, indicating that several BP components are glycosylated—or by treating with increasing levels of PNGase F (Peptide-N-Glycosidase F) and immunostaining with the appropriate antibody. Both BMP-2 and BMP-7 showed some degree of glycosylation, but appeared to have some level of protein that was resistant to PNGase F, as well. Functional activity of PNGase F- and sialadase-treated samples was assayed by explant mass and ALP score, and it was observed that glycosylation is required for full activity.

In summary, BMPs 2, 3 and 7 are modified by phosphorylation (˜33%) and glycosylation (50%). These post-translation modifications affect protein morphogenic activity.

Regardless of the precise components of the mixture, administration of the mixture can be by any route which allows the delivery of the growth factors in active form to the kidney. Preferably, the mixture is administered subcutaneously, intramuscularly, or intravenously. Administration of the mixture via such routes will be a routine matter to one of ordinary skill in the art.

The mixture is administered at a dosage sufficient to treat renal injury. The dosage is preferably less than about 10 g/kg body weight per day, more preferably less than about 1 g/kg body weight per day, even more preferably less than about 0.1 g/kg body weight per day, most preferably less than about 0.01 g/kg body weight per day. The dosage can be provided either in discrete administrations (e.g. injections performed once, twice, three times, etc. per day), or in a continuous administration (such as can be provided by a continuous pump, intravenous drip, or similar apparatus).

Preferably, if the mixture is administered to treat a preexisting renal injury, the treatment regimen is begun as soon as possible after renal injury. If the mixture is administered prophylactically, the treatment regimen can be begun at any time before renal injury occurs.

The duration of the treatment regimen can be for any length of time, preferably until the renal injury is reduced or eliminated. Typically, the treatment regimen will have a duration of about 7 days to about 14 days after renal injury.

The method of the present invention can be used to treat any mammal. Preferably, the mammal is a human. However, the method is also applicable to veterinary treatment of other mammals, such as pets (e.g. dogs, cats), livestock (e.g. horses, cattle, sheep, goats), research mammals, and zoo mammals, among others.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Example 1. In vitro cell culture experiments

BP, comprising BMP-2, BMP-3, BMP-7, TGF-β, and FGF, was prepared from bovine bone according to a method substantially the same as described in Poser et al., U.S. Pat. No. 5,290,763, and characterized as described above.

A culture of human renal tubular epithelial cells was prepared. Varying concentrations of BP, ranging from 0.0 μg/mL culture to 10.0 μg/mL culture, were added, and after 24 hours at 37° C., the concentration of cells/mL was determined. The results are as follows.

TABLE 1
BP-induced proliferation of human renal tubular epithelial cells

| BP, μg/mL culture | Cells/mL, ×10⁵ |
|---|---|
| 0.0 | 1.8 |
| 0.1 | 1.7 |
| 1.0 | 2.7 |
| 5.0 | 2.9 |
| 10.0 | 2.0 |

As these results indicate, BP at levels of 1.0 and 5.0 μg/mL culture induced roughly 50%-65% higher cell counts than the control without added BP. Accordingly, BP is capable of inducing proliferation of human renal tubular epithelial cells in vitro.

Example 2. Effect of BP on TGF-β levels in vitro

It has been observed that exposure of renal tubular cells to high levels of glucose induces the production of TGF-β. TGF-β has been implicated as inducing fibrosis in the kidney. To test the effect of BP on TGF-β production, renal tubular cells were exposed in vitro to high levels of glucose (4× or 6× the usual concentration of 1.297 g/L, i.e. 6× glucose=7.782 g/L and 4×=5.188 g/L), in the presence or absence of BP. BP was as described in Example 1.

The results are shown in Table 2.

TABLE 2
Effect of BP on TGF-β levels

| Glucose concentration | BP, μg | TGF-β, pg/mL |
|---|---|---|
| 6x | 0.0 | 38 |
| 4x | 0.0 | 13 |
| 6x | 5.0 | 1 |
| 4x | 1.0 | 11 |

These results indicate that BP levels of from 1.0 μg to 5.0 μg inhibited the overexpression of TGF-β under high levels of glucose. This suggests that BP can be used to treat renal injury with minimal risk of kidney fibrosis.

Example 3. In vivo effects of BP in treating renal injury

The effectiveness of BP in treating an animal model of acute renal injury was tested according to the following example. BP was as described in Example 1 above. Rats underwent renal ischemia by clamping both renal arteries for time intervals of 30-50 min to induce a reversible injury to the kidneys. Renal function was assessed by determining blood urea nitrogen (BUN) and mortality. Three groups were tested, with at least 4 animals treated with BP (10 g/kg body weight every 24 hr, beginning concurrently with induction of ischemia) and a control of at least 4 untreated animals in each group. Mortality was observed after about 48 hours, with the results given as follows.

TABLE 3
Effect of BP on mortality rates after 30-50 min renal ischemia

| Duration of Ischemia | Survived/Total (control) | Survived/Total (BP) |
|---|---|---|
| 50 min | 0/4 | ¾ |
| 40 min | 5/10 | 12/16 |
| 30 min | 2/4 | 4/4 |

As seen from Table 3, 50 min of ischemia proved 100% fatal to the control group, and lesser durations of ischemia resulted in 50% mortality. In the BP treated group, by contrast, mortality at 50 min of ischemia was only 25%; the same mortality rate was observed for 40 min of ischemia. Mortality in the treated group was 0% at 30 min of ischemia.

That the reduced mortality was a result of BP treatment of the renal injury is shown by BUN levels measured daily in control and BP-treated animals, as shown in the following table.

TABLE 4
BUN levels in control and BP-treated animals

| Day | BUN level, control | BUN level, BP-treated |
|---|---|---|
| 0 (before treatment) | 20 | 20 |
| 1 | 125 | 90 |
| 2 | 135 | 50 |
| 3 | 100 | 45 |
| 4 | 80 | 45 |
| 5 | 65 | 40 |

These results show that blood urea nitrogen levels had a lower maximum and a faster return to baseline levels in BP-treated animals than in control animals. This indicates that kidney function was improved in the BP-treated animals relative to the controls.

Example 4. Characterization of BP

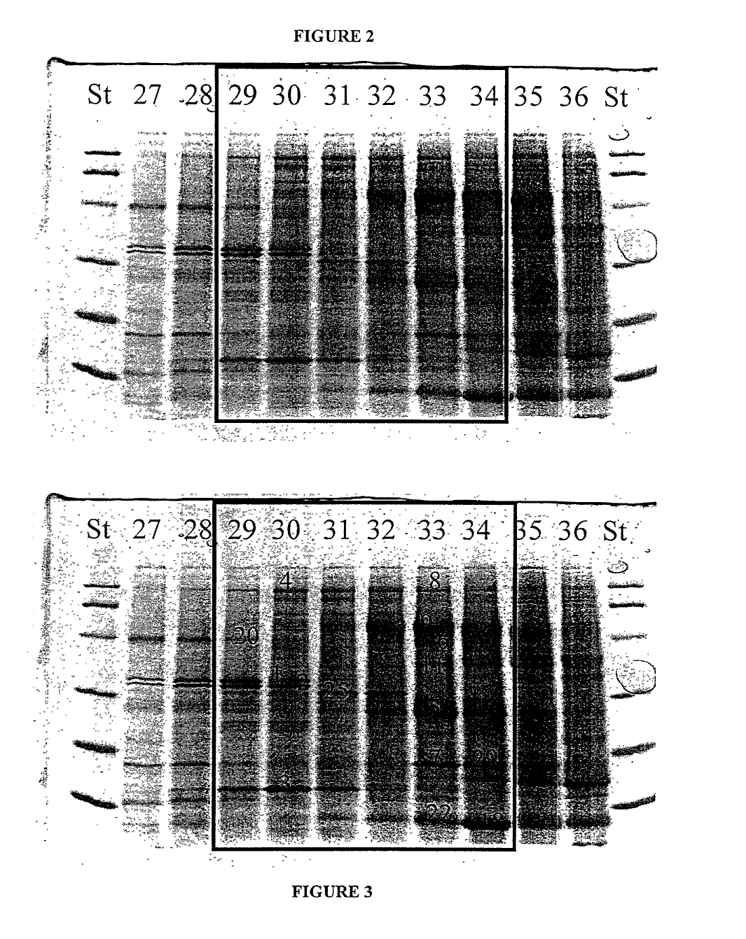

BP has been partially characterized as follows: high performance liquid chromatography (“HPLC”) fractions have been denatured, reduced with DTT, and separated by sodium dodecyl sulfate polyacrylamide gel electrophoresis (SDS-PAGE). One minute HPLC fractions from 27 to 36 minutes are shown in FIG. 2. Size standards (ST) of 14, 21, 31, 45, 68 and 97 kDa were obtained as Low Range size standards from BIORAD(tm) and are shown at either end of the coomassie blue stained gel. In the usual protocol, HPLC fractions 29 through 34 are pooled to produce BP (see boxes, FIGS. 2 and 3), as shown in a similarly prepared SDS-PAGE gel in FIG. 17B.

The various components of BP were characterized by mass spectrometry and amino acid sequencing of tryptic fragments where there were sufficient levels of protein for analysis. The major bands in the ID gel (as numerically identified in FIG. 3) were excised, eluted, subjected to tryptic digestion and the fragments were HPLC purified and sequenced. The sequence data was compared against known sequences, and the best matches are shown in FIGS. 15A-B. These identifications are somewhat tentative in that only portions of the entire proteins have been sequenced and, in some cases, there is variation between the human and bovine analogs for a given protein.

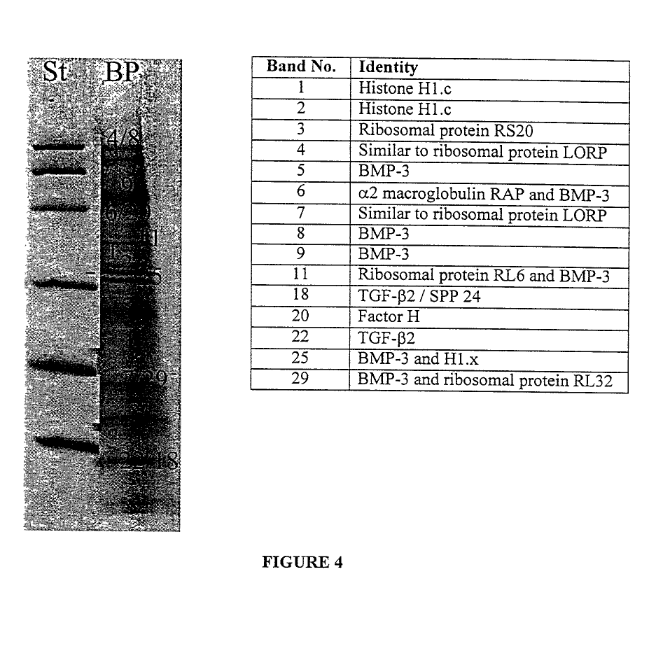

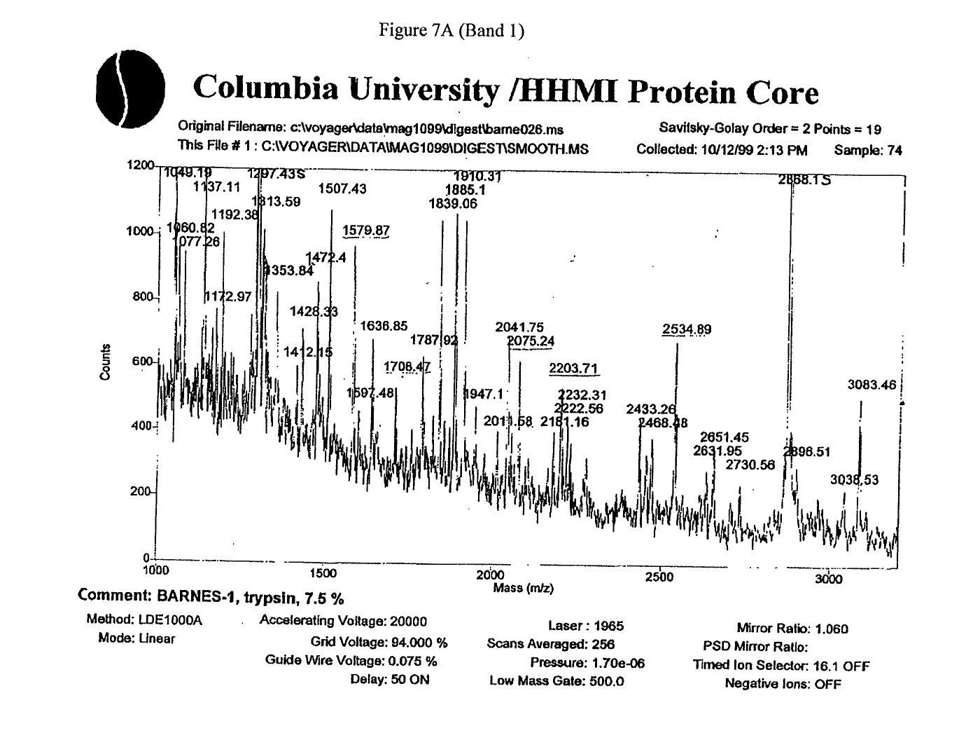

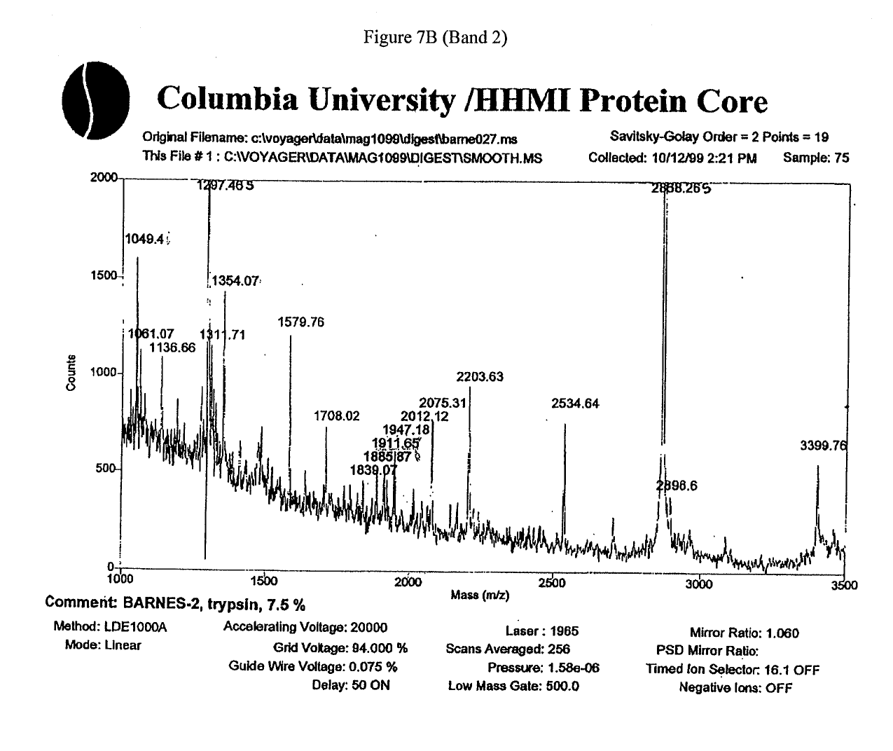

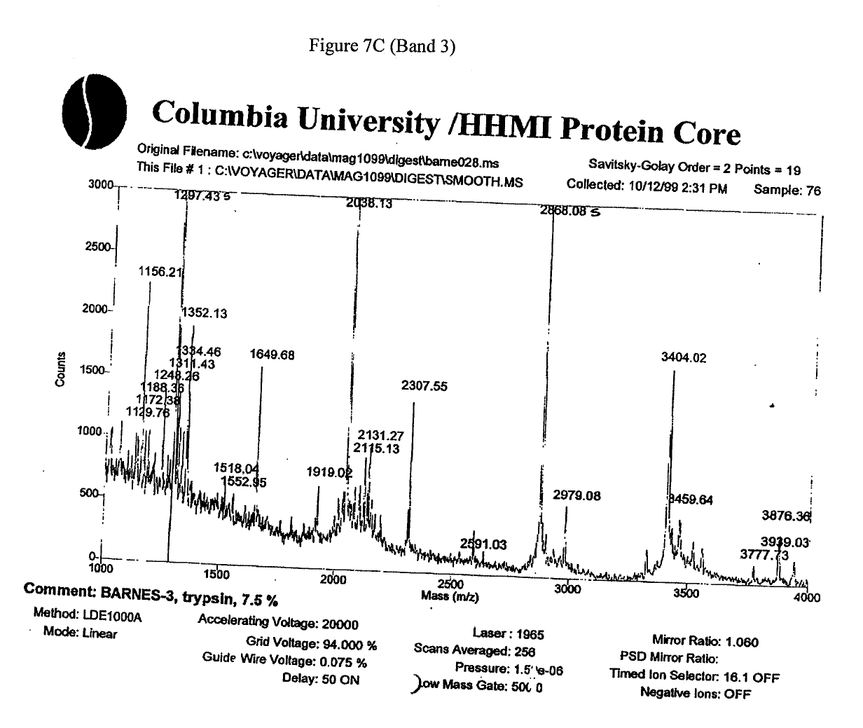

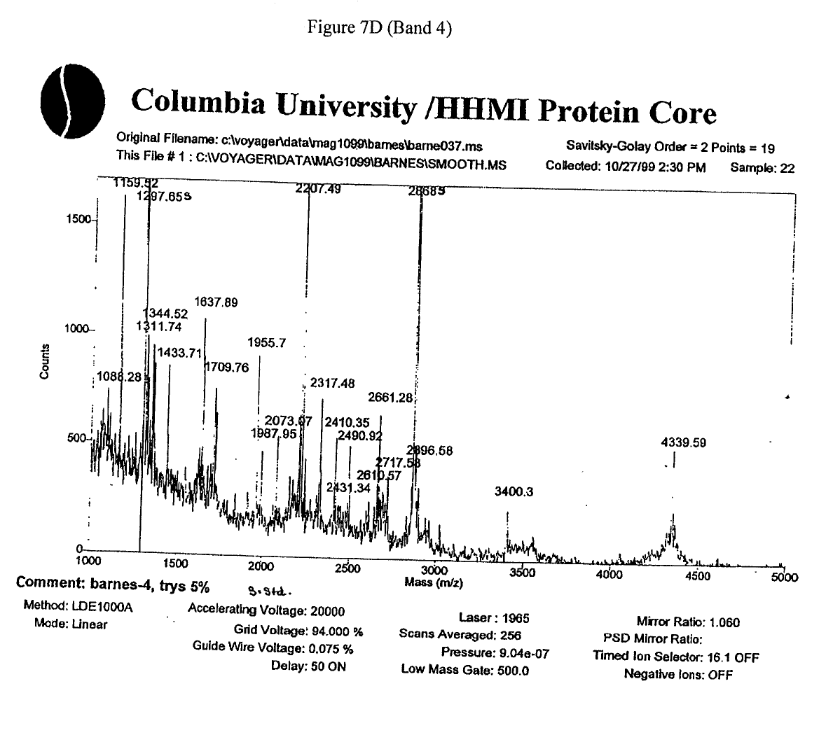

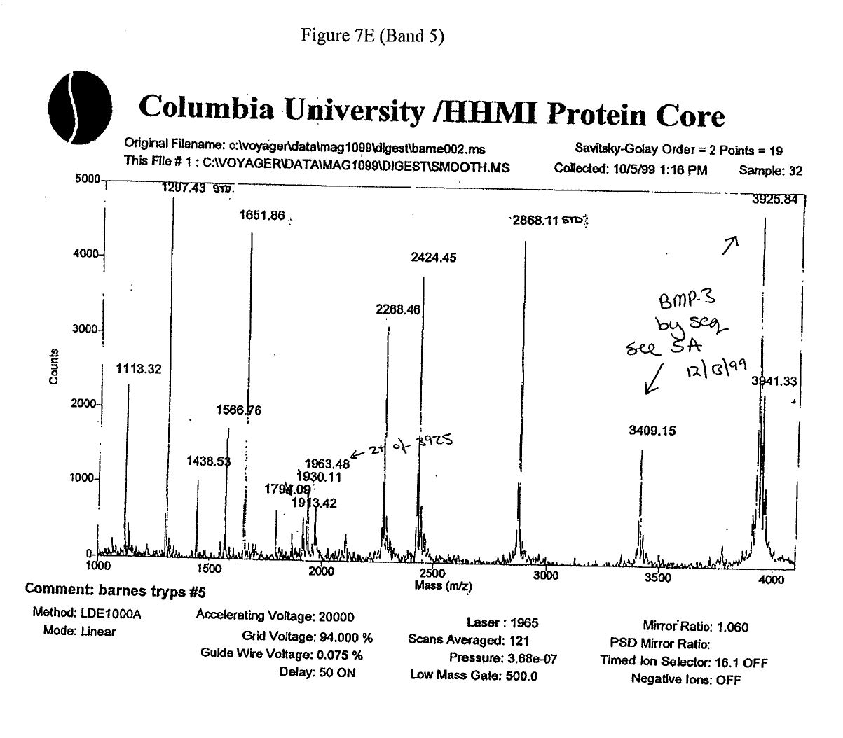

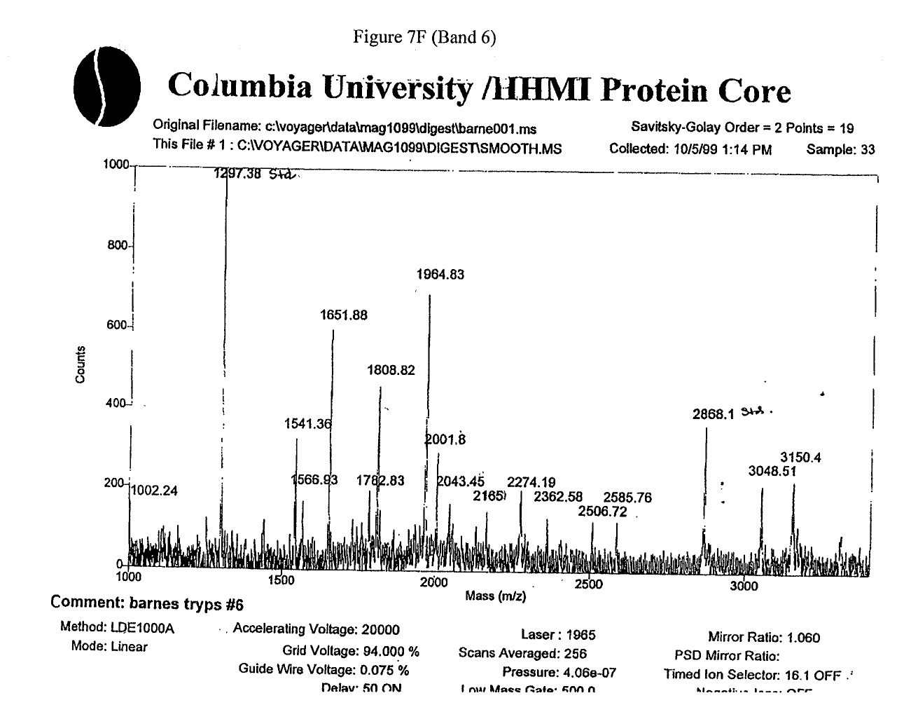

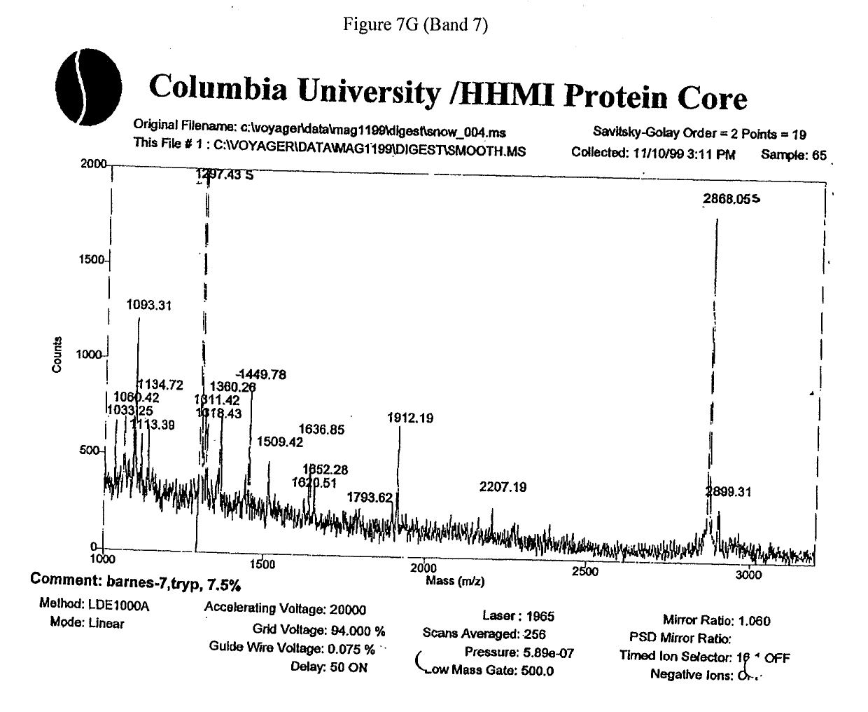

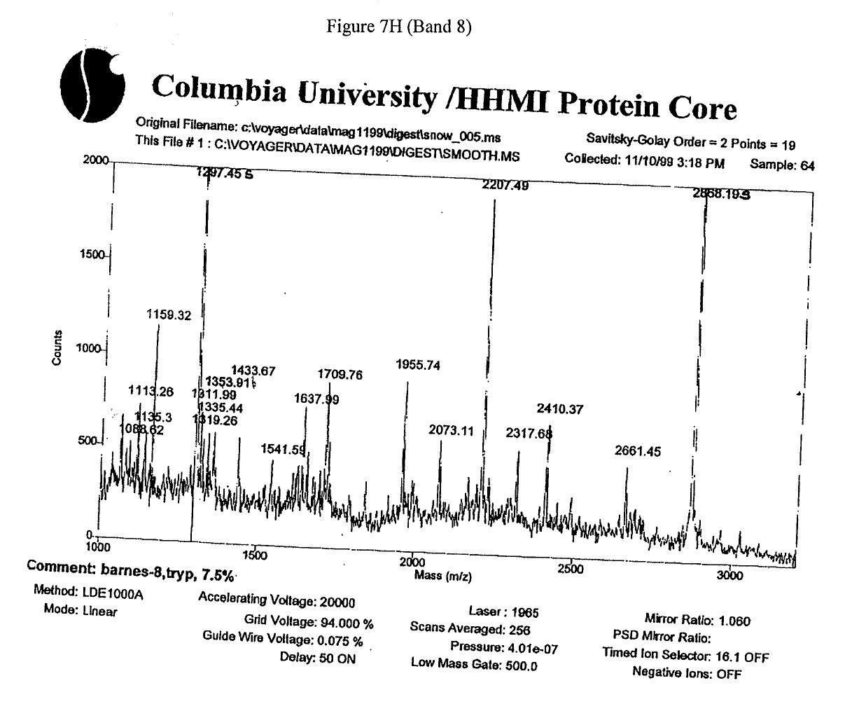

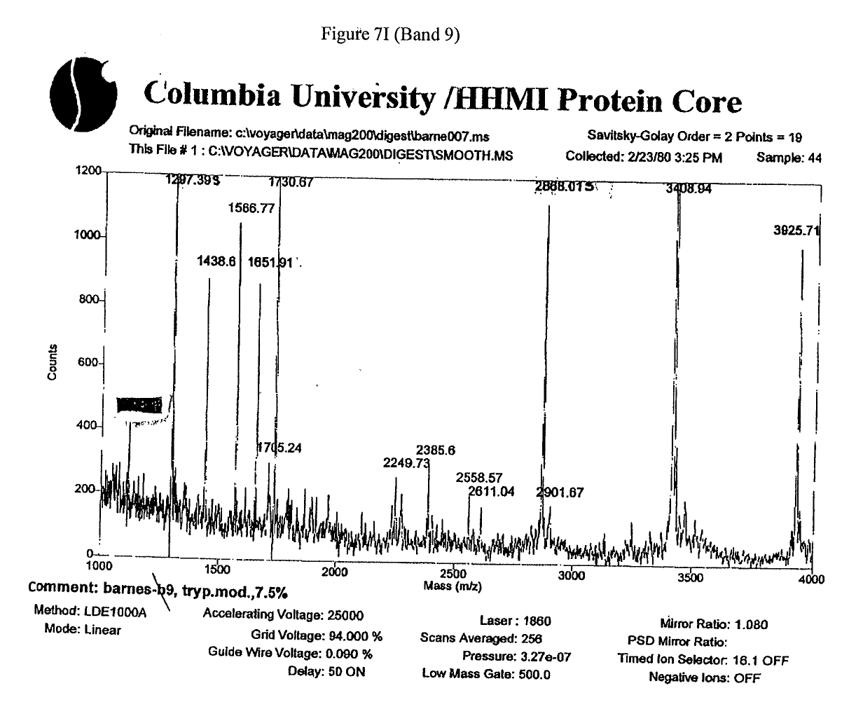

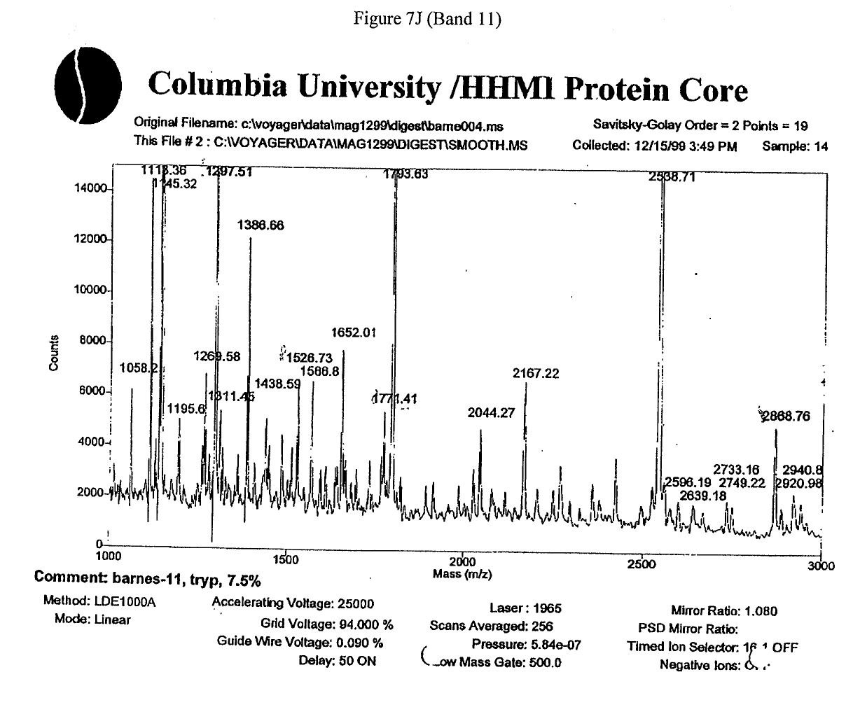

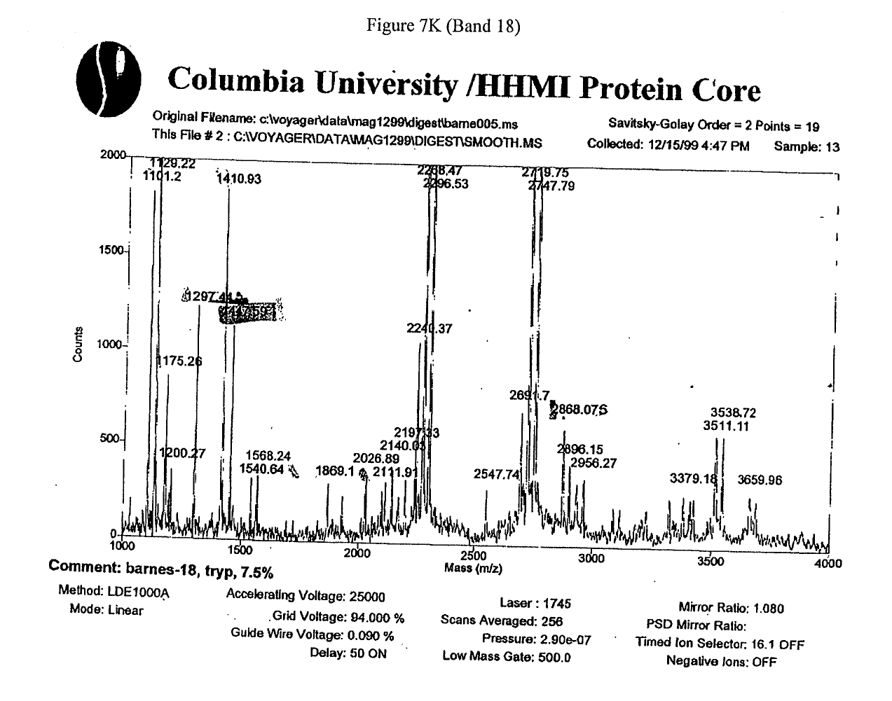

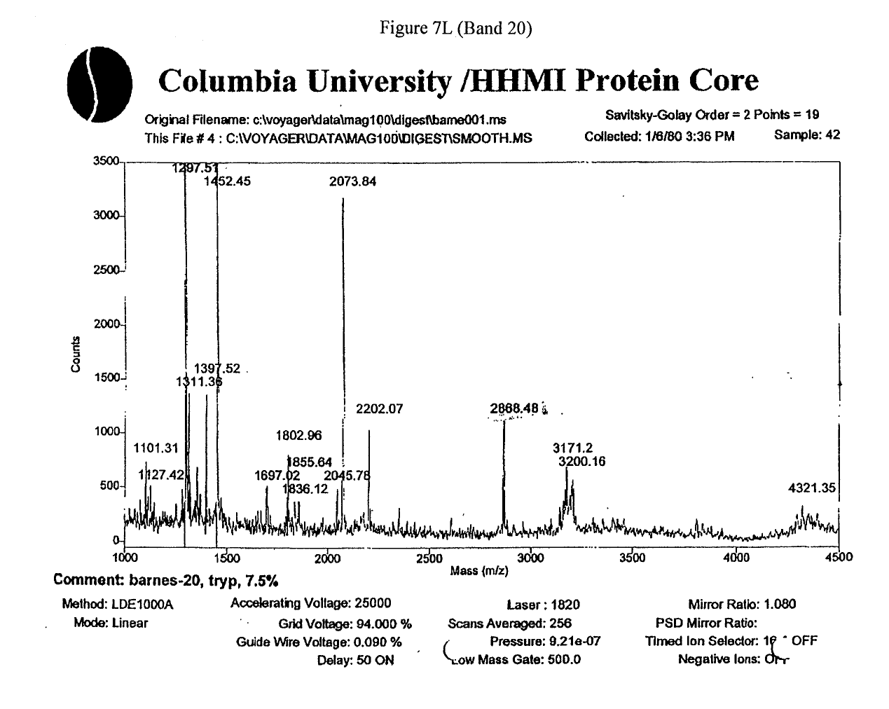

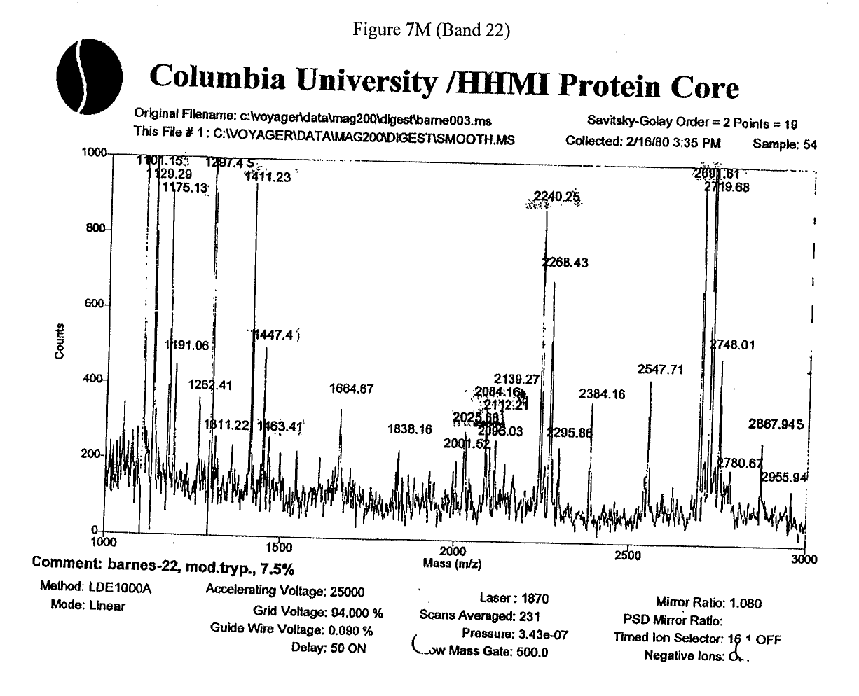

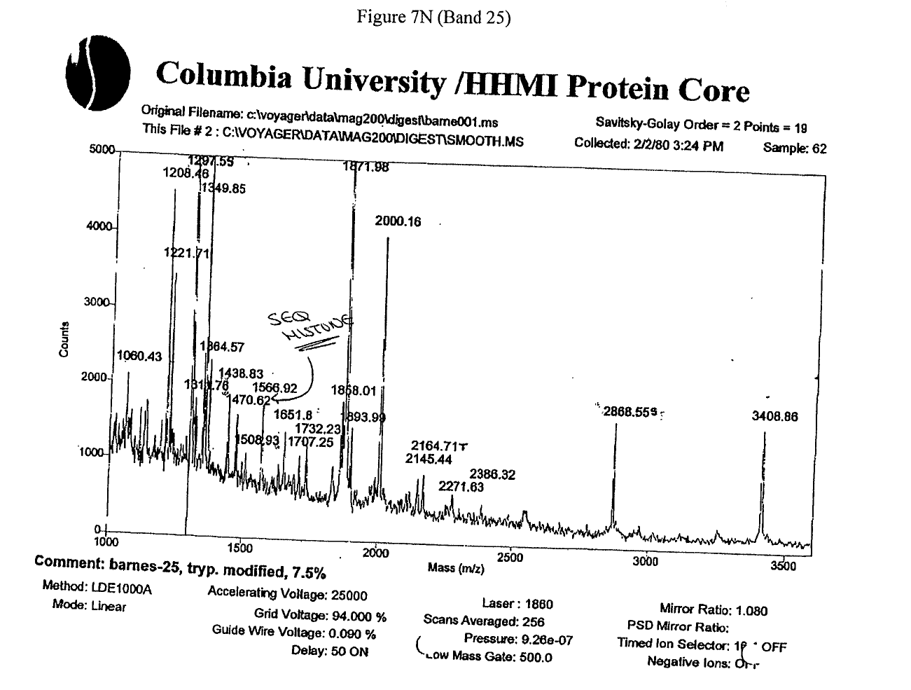

The same tryptic protein fragments were analyzed by mass spectrometry and the mass spectrograms are shown in FIGS. 7A-O. The tabulated results and homologies are shown in FIGS. 16A-F which provides identification information for the bands identified in FIGS. 3-4. As above, assignment of spot identity may be tentative based on species differences and post translational modifications. A summary of all protein identifications from ID gels is shown in FIG. 4.

The identified protein components of BP, as described in FIGS. 15A-B, 16A-F and 19A-D, were quantified as shown in FIGS. 17A and 17B. FIG. 17B is a stained SDS-PAGE gel of BP and FIG. 17A represents a scanning densitometer trace of the same gel. The identified proteins were labeled and quantified by measuring the area under the curve. These results are presented in FIG. 18 as a percentage of the total peak area.

Thus, there are 11 major bands in the BP SDS-PAGE gel representing about 60% of the protein in BP. The identified proteins fall roughly into three categories: the ribosomal proteins, the histones and growth factors, including bone morphogenic factors (BMPs). It is expected that the ribosomal proteins and histone proteins may be removed from the BP without loss of activity, since these proteins are known to have no growth factor activity. Upon this separation, the specific activity is expected to increase correspondingly.

Experiments are planned to confirm the hypothesis that the histone and ribosomal proteins may be removed from the BP with no resulting loss, or even an increase, in specific activity. Histones will be removed from the BP cocktail by immunoaffinity chromatography using either specific histone protein antibodies or a pan-histone antibody. The histone depleted BP (BP-H) will be tested as described above for wound healing and/or osteogenic activity. Similarly, the known ribosomal proteins will be stripped and the remaining mixture (BP-R) tested.

An SDS-PAGE gel of BP was also analyzed by Western immunoblot with a series of antibodies, as listed in FIG. 14. Visualization of antibody reactivity was by horse radish peroxidase conjugated to a second antibody and using a chemiluminescent substrate. Further, TGF-β1 was quantified using commercially pure TGF-β1 as a standard and was determined to represent less than 1% of the BP protein The antibody analysis indicated that each of the proteins listed in FIG. 14 is present in BP.

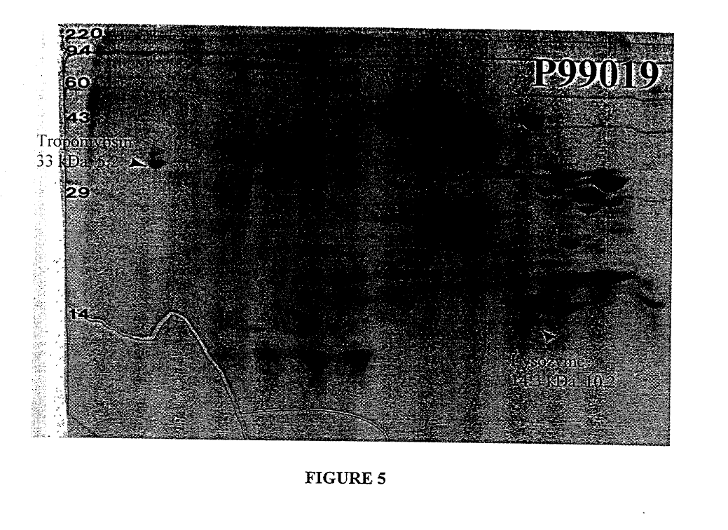

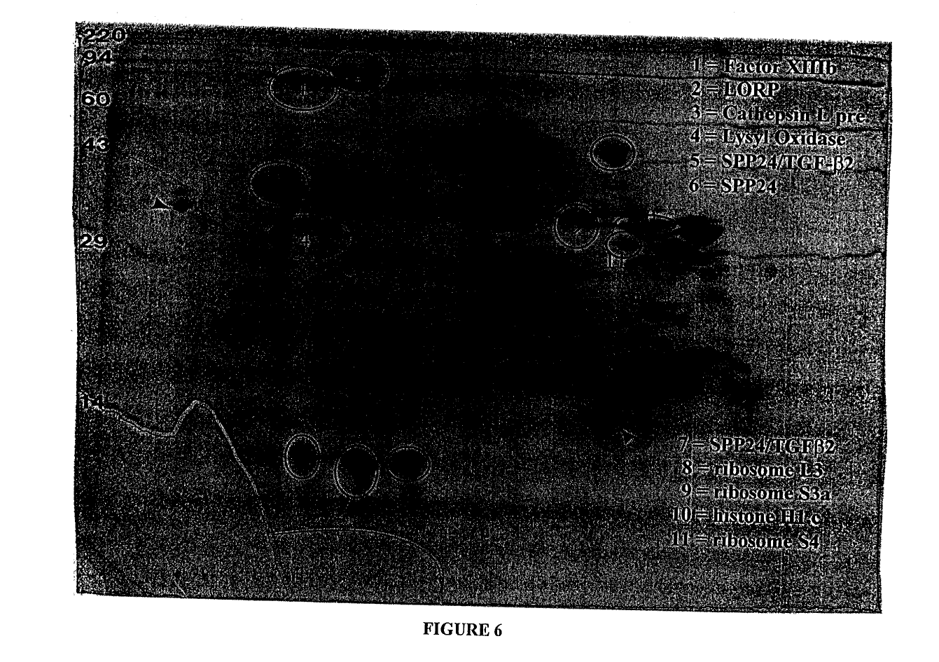

The BP was further characterized by 2-D gel electrophoresis, as shown in FIGS. 5-6. The proteins are separated in horizontal direction according to charge (pI) and in the vertical direction by size as described in two-dimensional electrophoresis adapted for resolution of basic proteins was performed according to the method of O'Farrell et al. (O'Farrell, P. Z., Goodman, H. M. and O'Farrell, P. H., Cell, 12: 1133-1142, 1977) by the Kendrick Laboratory (Madison, Wis.). Two-dimensional gel electrophoresis techniques are known to those of skill in the art. Nonequilibrium pH gradient electrophoresis (“NEPHGE”) using 1.5% pH 3.5-10, and 0.25% pH 9-11 ampholines (Amersham Pharmacia Biotech, Piscataway, N.J.) was carried out at 200 V for 12 hrs. Purified tropomyosin (lower spot, 33,000 KDa, pI 5.2), and purified lysozyme (14,000 KDa, pI 10.5 - 11) (Merck Index) were added to the samples as internal pI markers and are marked with arrows.

After equilibration for 10 min in buffer “0” (10% glycerol, 50 mM dithiothreitol, 2.3% SDS and 0.0625 M tris, pH 6.8) the tube gel was sealed to the top of a stacking gel which is on top of a 12.5% acrylamide slab gel (0.75 mm thick). SDS slab gel electrophoresis was carried out for about 4 hrs at 12.5 mA/gel.

After slab gel electrophoresis two of the gels were coomassie blue stained and the other two were transferred to transfer buffer (12.5 mM Tris, pH 8.8, 86 mM Glycine, 10% MeoH) transblotted onto PVDF paper overnight at 200 mA and approximately 100 volts/two gels. The following proteins (Sigma Chemical Co., St. Louis, Mo.) were added as molecular weight standards to the agarose which sealed the tube gel to the slab gel: myosin (220,000 KDa), phosphorylase A (94,000 KDa), catalase (60,000 KDa), actin (43,000 KDa), carbonic anhydrase (29,000 KDa) and lysozyme (14,000 KDa). FIG. 5 shows the stained 2-D gel with size standards indicated on the left. Tropomyosin (left arrow) and lysozyme (right arrow) are also indicated.

The same gel is shown in FIG. 6 with several identified proteins indicated by numbered circles. The proteins were identified by mass spectrometry and amino acid sequencing of tryptic peptides, as described above. The identity of each of the labeled circles is provided in the legend of FIG. 6 and the data identifying the various protein spots is presented in FIGS. 19A-D.

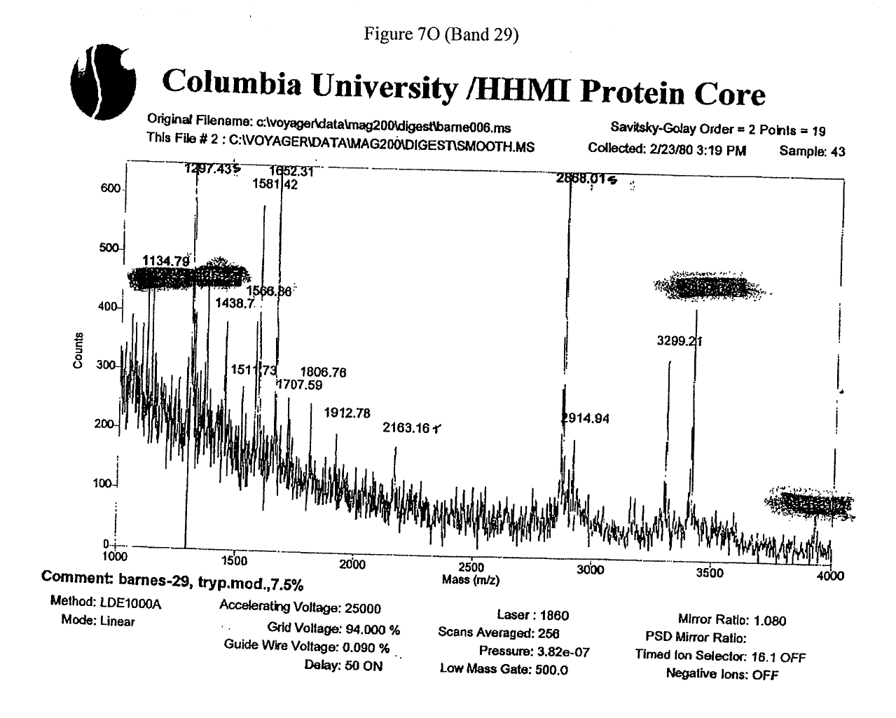

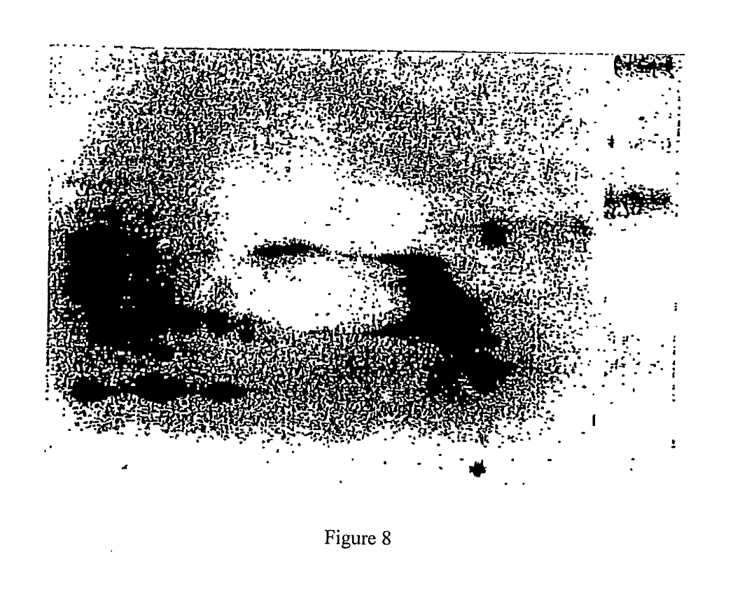

Because several of the proteins migrated at more than one size (e.g., BMP-3 migrating as 6 bands) investigations were undertaken to investigate the extent of post-translation modification of the BP components. Phosphorylation was measured by anti-phosphotyrosine immunoblot and by phosphatase studies. FIG. 8 shows a 2-D gel, electroblotted onto filter paper and probed with a phosphotyrosine mouse monoclonal antibody by SIGMA (# A-5964). Several proteins were thus shown to be phosphorylated at one or more tyrosine residues.

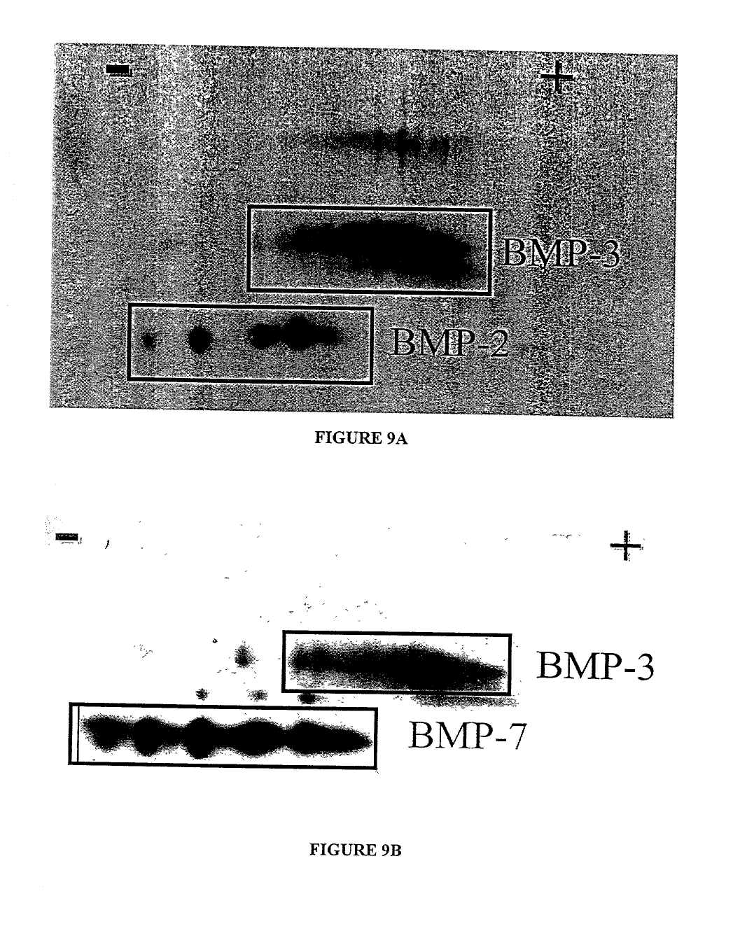

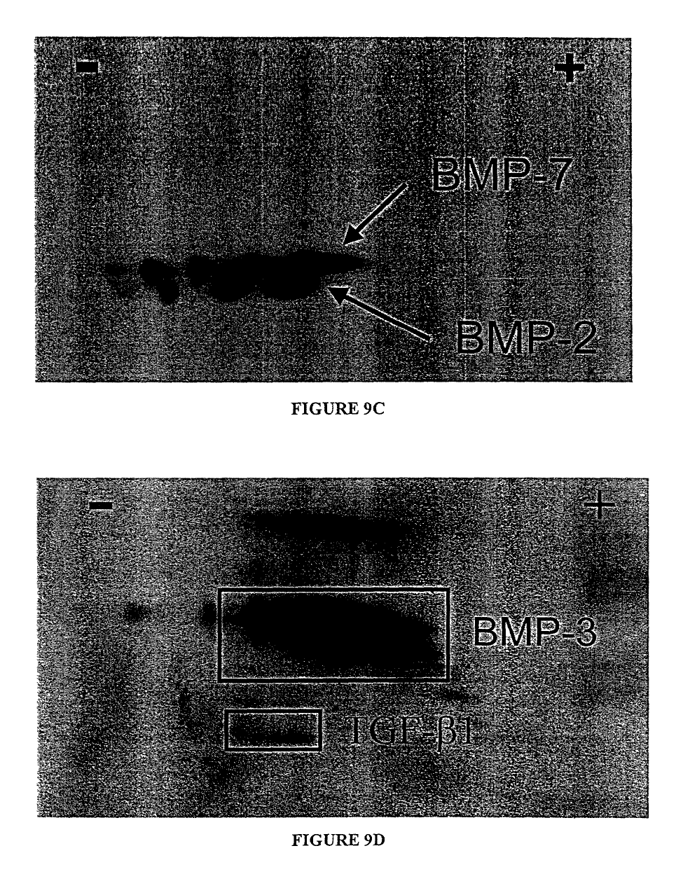

Similar 2-D electroblots were probed with BP component specific antibodies, as shown in FIGS. 9A-D. The filters were probed with BMP-2, BMP-3 (FIG. 9A), BMP-3, BMP-7 (FIG. 9B), BMP-7, BMP-2 (FIG. 9C), and BMP-3 and TGF-β1 (FIG. 9D). Each shows the characteristic, single-size band migrating at varying pI, as is typical of a protein existing in various phosphorylation states.

For the phosphatase studies, BP in 10 mM HCl was incubated overnight at 37° C. with 0.4 units of acid phosphatase (AcP). Treated and untreated samples were added to lyophilized discs of type I collagen and evaluated side by side in the subcutaneous implant rat bioassay, as previously described in U.S. Pat. Nos. 5,290,763, 5,563,124 and 5,371,191. Briefly, 10 μg of BP in solution was added to lyophilized collagen discs and the discs implanted subcutaneously in the chest of a rat. The discs were then recovered from the rat at 2 weeks for the alkaline phosphotase (“ALP”—a marker for bone and cartilage producing cells) assay or at 3 weeks for histological analysis. For ALP analysis of the samples, the explants were homogenized and levels of ALP activity measured using a commercial kit. For histology, thin sections of the explant were cut with a microtome, and the sections stained and analyzed for bone and cartilage formation.

Both native- and phosphatase-treated BP samples were assayed for morphogenic activity by mass of the subcutaneous implant (explant mass) and ALP score. The results showed that AcP treatment reduced the explant mass and ALP score from 100% to about 60%. Thus, phosphorylation is important for BP activity.

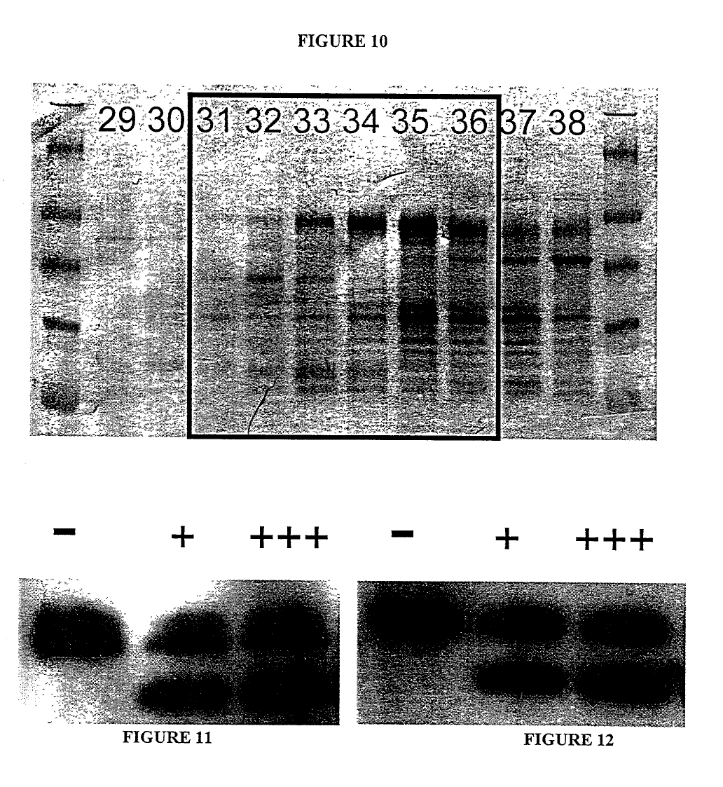

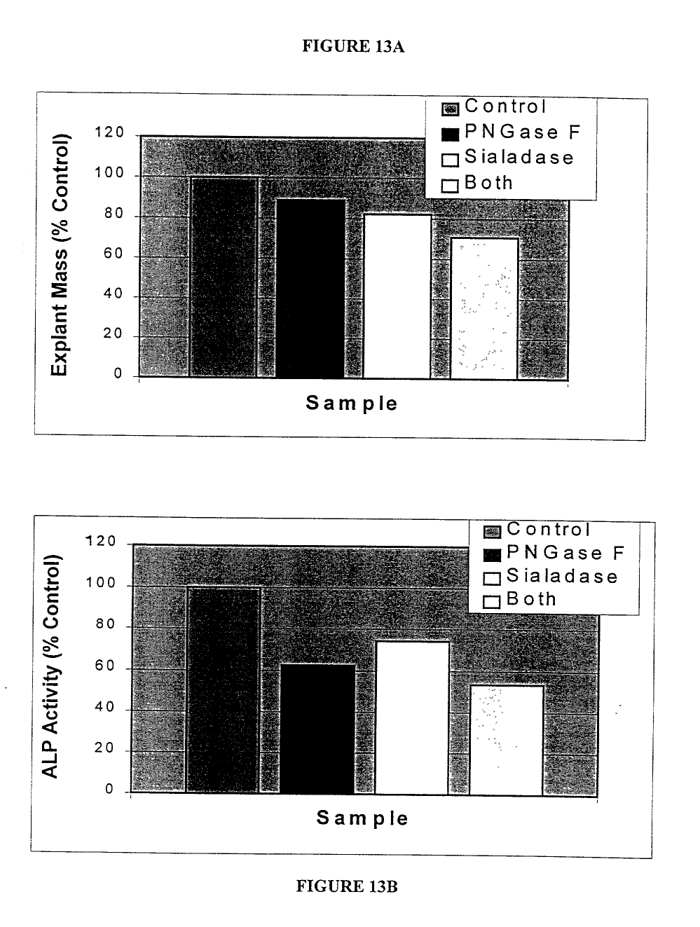

The BP was also analyzed for glycosylation. FIG. 10 shows an SDS-PAGE gel stained with periodic acid schiff (PAS)—a non-specific carbohydrate stain, indicating that several of the BP components are glycosylated (starred protein identified as BMP-3). FIGS. 11-12 show immunodetection of two specific proteins (BMP-7, FIG. 11 and BMP-2, FIG. 12) treated with increasing levels of PNGase F (Peptide-N-Glycosidase F). Both BMP-2 and BMP-7 show some degree of glycoslyation in BP, but appear to have some level of protein resistant to PNGase F as well (plus signs indicate increasing levels of enzyme). Functional activity of PNGase F and sialadase treated samples were assayed by explant mass and by ALP score, as shown in FIGS. 13A and 13B which shows that glycosylation is required for full activity.

In summary, BMPs 2, 3 and 7 are modified by phosphorylation and glycosylation. These post-translation modifications affect protein morphogenic activity, 33% and 50% repectively, and care must be taken in preparing BP not to degrade these functional derivatives.

The methods disclosed and claimed herein can be made and executed without undue experimentation in light of the present disclosure. While the compositions and methods of this invention have been described in terms of preferred embodiments, it will be apparent to those of skill in the art that variations may be applied to the method and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is

1. A method of treating renal injury in a mammal, comprising:
administering to the mammal a mixture of growth factors comprising at least two growth factors selected from the group consisting of bone morphogenic protein-2 (BMP-2), bone morphogenic protein-3 (BMP-3), bone morphogenic protein-4 (BMP-4), bone morphogenic protein-5 (BMP-5), bone morphogenic protein-6 (BMP-6), bone morphogenic protein-7 (BMP-7), transforming growth factor β1 (TGF-β1, transforming growth factor β2 (TGF-β2, transforming growth factor β3 (TGF-β3, and fibroblast growth factor 1 (FGF-1).

2. The method of claim 1, wherein the mammal is a human.

3. The method of claim 1, wherein the mixture is administered subcutaneously, intramuscularly, or intravenously.

4. The method of claim 1, wherein the mixture is administered discretely or continuously.

5. The method of claim 1, wherein the mixture further comprises a growth factor selected from insulin-like growth factor-1 (IGF-1), epidermal growth factor (EGF), hepatocyte growth factor (HGF), transforming growth factor α (TGF-α or platelet-derived growth factor (PDGF).

6. The method of claim 1, wherein the mixture further comprises a preservative or an adjuvant.

7. The method of claim 1, wherein the mixture comprises BMP-2, BMP-3, BMP-7, TGF-β, and FGF.

8. The method of claim 1, wherein the mixture is derived by
(i) grinding mammalian bone, to produce ground bone;
(ii) cleaning the ground bone, to produce cleaned ground bone;
(iii) demineralizing the cleaned ground bone, to produce demineralized cleaned ground bone;
(iv) extracting protein from the demineralized cleaned ground bone using a protein denaturant; to yield extracted protein;
(v) ultrafiltering the extracted protein to separate out high molecular weight proteins;
(vi) ultrafiltering the extracted protein to separate out low molecular weight proteins;
(vii) transferring the extracted protein to a non-ionic denaturant;
(viii) subjecting the extracted protein to an anion exchange process;
(ix) subjecting the extracted protein to a cation exchange process; and
(x) subjecting the extracted protein to a reverse phase HPLC process.

9. The method of claim 8, wherein the mammalian bone is bovine bone.

10. The method of claim 8, wherein the amino acid composition of the mixture is about 23.4 mole % ASP(+ASN) and GLU(+GLN); about 13.5 mole % SER and THR; about 40.0 mole % ALA, GLY, PRO, MET, VAL, ILE, and LEU; about 6.8 mole % TYR and PHE; and about 16.6 mole % HIS, ARG, and LYS.

11. The method of claim 8, wherein the mixture comprises at least about 19% total protein by weight BMP-3, less than about 6% total protein by weight TGF-β2less than about 1% total protein by weight TGF- β1.
